(19) United States
(12) Patent Application Publication
(10) Pub. No.: US 2018/0011346 A1
(43) Pub. Date: Jan. 11, 2018

(54) PROBE FOR TARGETING AND MANIPULATING MITOCHONDRIAL FUNCTION USING QUANTUM DOTS

(71) Applicant: Nanoco Technologies Ltd., Manchester (GB)

(72) Inventor: Imad Naasani, Manchester (GB)

(21) Appl. No.: 15/637,899

(22) Filed: Jun. 29, 2017

Related U.S. Application Data
(60) Provisional application No. 62/358,332, filed on Jul. 5, 2016.

(51) Int. Cl. G02F 1/017; A61K 9/14; A61K 31/275; A61K 31/66

(52) U.S. Cl. CPC G02F 1/017; A61K 31/66; A61K 9/141; A61K 31/275; G02F 2001/01791

(57) ABSTRACT

The present disclosure relates to quantum dot nanoparticles useful for targeting and manipulating mitochondrial function, and to methods of targeting and manipulating mitochondrial function using such quantum dot nanoparticles.

### FIELD OF THE INVENTION

Embodiments disclosed herein relates to quantum dot nanoparticles useful for targeting and manipulating mitochondrial function, and to methods of targeting and manipulating mitochondrial function using such quantum dot nanoparticles.

### BACKGROUND OF THE INVENTION

Mitochondria are cellular organelles with many roles, including energy production, heat generation, hormone synthesis, regulation of metabolism and calcium, release of the canonical enzyme cytochrome c oxidase (COX), apoptosis, production of reactive oxygen species (ROS) homeostasis and aging. Recently, targeting mitochondria has been considered for various disease management protocols, including sepsis, acute organ failure in end of life stages, cancer, Alzheimer, and diabetes. See, e.g., Karu et al., Life, 62(8), 607-610, 2010; Singer et al., Virulence, 5(1), 66-72, 2014; Wu et al., Antioxidants & Redox Signaling, 20(5), 733-745, 2014; Pathania et al., Adv. Drug Delivery Rev., 61, 1250-1275, 2009; and D'Souza et al., Biochimica et Biophysica Acta, 1807, 689-696, 2011.

Recent studies suggest that nanoparticles have a tendency for specific accumulation in subcellular organelles primarily lysosomes. See, e.g., Frohlich, International Journal of Nanomedicine, Oct. 31, 2012, 5577-5591. Mitochondria have also been found to accumulate nanoparticles. See, e.g., D'Souza et al., Biochimica et Biophysica Acta 1807 (2011) 689-696.

Studies have also shown that the enzyme cytochrome c oxidase (COX) is considered as a photo-acceptor and photo-signal transducer in the region of visible and near infra-red (NIR). Photo-irradiation causes an increase in COX activity that leads to a cascade of reactions which can alter cellular homeostasis and increase the production of adenosine triphosphate (ATP), reactive oxygen species (ROS), nitric oxide (NO), and intracellular calcium (iCa²⁺). See, e.g., Karu et al., Multiple roles of cytochrome c oxidase in mammalian cells under action of red and IR-A radiation, Life, 62(8), 607-610, 2010 and Tafur et al., Photomedicine and Laser Surgery, Volume 26, Number 4, 2008 323-28.

There has been substantial interest in the preparation and characterization of particles with dimensions, for example in the range 2-50 nm, often referred to as quantum dots (QDs) or nanocrystals. These studies have occurred mainly due to the size-tunable electronic properties of these materials that may be exploited in, for example, optical and electronic devices, solar cells, catalysis, light-emitting diodes and general space lighting. QDs have widely been investigated for their unique optical, electronic and chemical properties, which originate from “quantum confinement effects”; as the dimensions of a semiconductor nanoparticle are reduced below twice the Bohr radius, the energy levels become quantized, giving rise to discrete energy levels. The band gap increases with decreasing particle size, leading to size-tunable optical, electronic and chemical properties, such as size-dependent photoluminescence.

The advent of such quantum dots as bright and highly absorptive fluorophores provides an opportunity for targeting and manipulating mitochondrial function.

There is a need for new methods to target and manipulate mitochondrial function.

### SUMMARY OF THE INVENTION

Embodiments disclosed include quantum dot nanoparticles that may be used for targeting mitochondrial function. By manipulating the optical and/or photovoltaic properties of such quantum dot nanoparticles, certain mitochondrial functions (such as, e.g., COX activity or membrane potential) may be modulated for detection and labeling purposes and/or for therapeutic purposes.

In one aspect, the present invention relates to a quantum dot nanoparticle useful for targeting mitochondrial function.

In one embodiment, the quantum dot nanoparticles described herein are conjugated to a functional ligand to allow metabolic activities in mitochondria to be modulated (e.g., slowed or stopped).

In one embodiment, the quantum dots nanoparticles are conjugated to a hexokinase (HK) inhibitor, such as 2-deoxyglucose (2DG). The enzyme HK is responsible for glycolysis and is associated with the mitochondrial membrane. Glycolysis is a main source for energy in cancer cells. The quantum dot nanoparticles can therefore have three functions, either separate or in combination: i) delivery of a glycolysis inhibitor, such as, for example, a hexokinase (HK) inhibitor, such as 2-deoxyglucose (2DG); ii) activation of COX, and iii) labeling.

In one embodiment, the nanoparticle is linked (e.g., covalently bonded or physically bonded (charge or Van de Waals) to the functional ligand via an amide, ester, thioester, or thiol anchoring group directly on the inorganic surface of the quantum dot nanoparticle or on the organic corona layer that is used to render the nanoparticles water soluble and biocompatible. The water soluble QD nanoparticle in certain embodiments includes a core of one semiconductor material and at least one shell of a different semiconductor molecule in some embodiments while in other embodiments the water soluble QD nanoparticle includes an alloyed semiconductor material having a bandgap value that increases outwardly by compositionally graded alloying.

In certain embodiments the light responsive quantum dot (QD) includes water soluble QD nanoparticles having a ligand interactive agent and a surface modifying ligand. The water soluble QD nanoparticle may be formed by chemical addition of the ligand interactive agent and the surface modifying ligand to the QD in a solution comprising hexamethoxymethylmelamine. In particular embodiments the ligand interactive agent is a C₈₋₂₀ fatty acid and esters thereof, while the surface modifying ligand is a monomethoxy polyethylene oxide.

In certain embodiments, the water soluble nanoparticles include capping ligands that are able to bind to methylation specific binding ligands. In certain embodiments the capping ligand is selected from the group consisting of: thiol, carboxyl, amine, phosphine, phosphine oxide, phosphonic acid, phosphinic acid, imidazole, OH, thio ether, and calixarene groups.

In another embodiment, the nanoparticle is bound to the functional ligand by ion pairing or Van Der Waals interactions. In one embodiment, the nanoparticle is covalently linked to the functional ligand via an amide bond.

In one embodiment, the quantum dot nanoparticle conjugate comprises: a quantum dot comprising a core semiconductor material, and an outer layer, wherein the outer layer comprises a corona of organic coating (a functionalization organic coating) to render the particles water soluble and bio compatible, and a functional ligand to allow metabolic activities in mitochondria to be modulated.

In one embodiment, any of the quantum dot nanoparticles described herein are surface modified (e.g., physically or chemically coated and/or charge modified) to enhance accumulation of the quantum dot in the vicinity or within mitochondria. Suitable coatings include, for example, mitochondriotopic ligands, such as, e.g., anti-mitochondria antibody sold by tebu-bio (cat. No. 909-301-D79), Anti HSP60 (T547) sold by tebu-bio (cat nos. BS1179-50 ul (50 ul) and BS1179-100 ul (100 ul)), Anti SOD1 sold by tebu-bio (cat no. MAB10394), Anti Grp75 clone S19-2 sold by tebu-bio (cat no. MAB6629), Anti Cytochrome c (H19) sold by tebu-bio (cat nos. BS1089-50 ul (50 ul) and BS1089-100 ul (100 ul)), triphenylphosphonium (TPP) and derivatives thereof. These mitochondrial specific ligands, once linked to the quantum dot nanoparticle using conjugation chemistry discussed below, ensures more specific accumulation of the quantum dot nanoparticle in the vicinity or within the mitochondria.

In one embodiment of any of the quantum dot nanoparticles described herein, the nanoparticle comprises a II-VI material, a III-V material, a material or any alloy thereof.

In an additional embodiment, any of the quantum dot nanoparticles described herein further comprise a cellular uptake enhancers, (cell-penetrating peptides (CPPs like TAT, RGD, or poly arginine), tissue penetration enhancers, (e.g., saponins, cationic lipids, Streptolysin O (SLO)), or a combination thereof. Examples of a cellular uptake enhancer include, for example, trans-activating transcriptional activators (TAT), Arg-Gly-Asp (RGD) tri-peptides, or poly arginine peptides. The ligand-nanoparticle conjugates can further comprise other known agents such as, for example, saponins, cationic liposomes or Streptolysin O, that can enhance cellular uptake.

In another aspect, a process for preparing a quantum dot nanoparticle according to any of the embodiments described herein. In one embodiment, the process comprises: i) coupling a nanoparticle with a functionalized ligand to give functionalized nanoparticle conjugate, wherein the nanoparticle comprises a quantum dot comprised of a core semiconductor material, and an outer layer, wherein the outer layer comprises a carboxyl group.

In one embodiment, the coupling step i) comprises (a) reacting a carboxyl group in the outer layer with a carbodiimide linker to activate the carboxyl group, and b) reacting the activated carboxyl group with a functionalized ligand (e.g., with an amine terminus on the ligand).

In an additional embodiment, the process further comprises: ii) purifying the quantum dot nanoparticle. In an additional embodiment, the process further comprises: iii) isolating the specific binding nanoparticle conjugate. In one embodiment, the process comprises steps i), ii) and iii).

In another aspect, the present invention provides a method of manipulating mitochondrial function. In one embodiment, the method comprises i) associating a quantum dot nanoparticle according to any of the embodiments described herein with mitochondria, and optionally ii) exciting the quantum dot nanoparticle with a light source thereby generating photons and/or voltaic current (voltaic energy). In one embodiment, the photons and/or voltaic current modulates the reactivity of mitochondrial function (such as, e.g., COX activity or membrane potential).

In one embodiment, the voltaic current generated (such as a micro voltage) stimulates the release of cytochrome C oxidase (COX), thereby inducing apoptosis. In another embodiment, the photons generated photo-modulate the activity of COX with, for example, light of 580-860 nm.

In another aspect, the present invention provides a method of modulating the activity of COX, or a subunit or derivative thereof (such as stimulating COX release). In one embodiment, the method comprises i) contacting a quantum dot nanoparticle according to any of the embodiments described herein with mitochondria, and optionally ii) exciting the quantum dot nanoparticle with a light source. In one embodiment, the COX is activated via absorbing photovoltaic energy. In another embodiment, the COX is activated via absorbing light energy.

In another aspect, the present invention provides a method of depolarizing a mitochondrial membrane (e.g., thereby inducing signaling cascades, e.g., apoptosis, leading to cell death). In one embodiment, the method comprises i) contacting a quantum dot nanoparticle according to any of the embodiments described herein with mitochondria, and ii) exciting the quantum dot nanoparticle with light source. In one embodiment, the mitochondrial membrane is depolarized via a photovoltaic effect.

In another aspect, the present invention provides a method of inhibiting glycolysis. In one embodiment, the method comprises i) contacting a quantum dot nanoparticle according to any of the embodiments described herein that is linked to a hexokinase (HK) inhibitor with mitochondria, and ii) exciting the quantum dot nanoparticle with a light source. In one embodiment, the HK inhibitor is 2-deoxyglucose (2DG).

### BRIEF DESCRIPTION OF THE DRAWINGS

FIG. 1 depicts cytoplasmic uptake of quantum dot nanoparticles in mouse embryonic stem cells after 5 hours, 24 hours and 48 hours. Nuclear regions were counter stained with blue/green using 4′-6-diamidino-2-phenylindole (DAPI), a fluorescent dye that binds strongly to A-T rich regions in DNA.

FIG. 2 depicts the proposed mechanism of interaction of quantum dot nanoparticles in mitochondria.

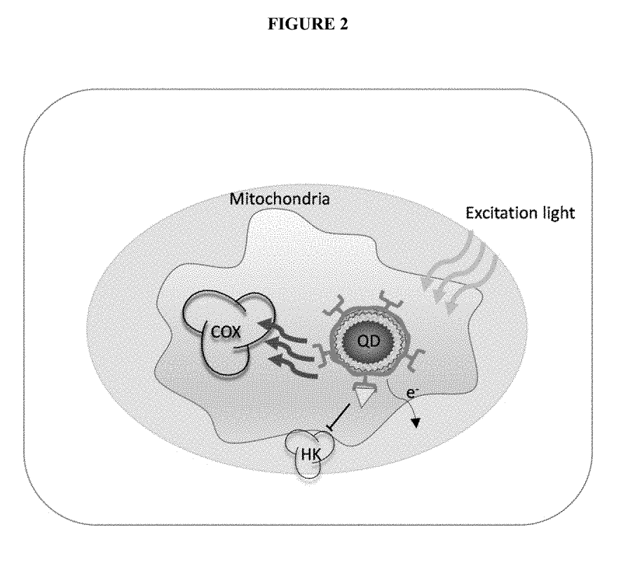

### DETAILED DESCRIPTION OF THE INVENTION

Disclosed herein are Quantum Dots (QDs) capable of associating with mitochondria and capable of emitting light to activate certain mitochondrial pathways. Disclosed herein are QDs that are conjugated with mitochondrial specific binding ligands that have the ability to come into proximity to a mitochondria sufficient to detect and manipulate mitochondrial function.

ABBREVIATIONS: The following abbreviations are used throughout this application:

DCC dicyclohexylcarbodiimide

DCM dichloromethane

DIC diisopropylcarbodiimide

EDC 1-ethyl-3-(-3-dimethylaminopropyl) carbodiimide hydrochloride

HMMM hexamethoxymethylmelamine

In(MA)₃ indium myristate

QD Quantum Dots

sulfo-NHS sulfo derivative of N-hydroxysuccinimide

SMCC succinimidyl 4-(N-maleimidomethyl)cyclohexane-1-carboxylate

(TMS)₃P tris(trimethylsilyl) phosphine

To facilitate the understanding of this invention, and for the avoidance of doubt in construing the claims herein, a number of terms are defined below. Terms defined herein have meanings as commonly understood by a person of ordinary skill in the areas relevant to the present invention. The terminology used to describe specific embodiments of the invention does not delimit the invention, except as outlined in the claims.

The terms such as “a,” “an,” and “the” are not intended to refer to a singular entity unless explicitly so defined, but include the general class of which a specific example may be used for illustration. The use of the terms “a” or “an” when used in conjunction with “comprising” in the claims and/or the specification may mean “one” but may also be consistent with “one or more,” “at least one,” and/or “one or more than one.”

The use of the term “or” in the claims is used to mean “and/or” unless explicitly indicated to refer to alternatives as mutually exclusive. Thus, unless otherwise stated, the term “or” in a group of alternatives means “any one or combination of” the members of the group. Further, unless explicitly indicated to refer to alternatives as mutually exclusive, the phrase “A, B, and/or C” means embodiments having element A alone, element B alone, element C alone, or any combination of A, B, and C taken together.

Similarly, for the avoidance of doubt and unless otherwise explicitly indicated to refer to alternatives as mutually exclusive, the phrase “at least one of” when combined with a list of items, means a single item from the list or any combination of items in the list. For example, and unless otherwise defined, the phrase “at least one of A, B and C,” means “at least one from the group A, B, C, or any combination of A, B and C.” Thus, unless otherwise defined, the phrase requires one or more, and not necessarily not all, of the listed items.

The terms “comprising” (and any form thereof such as “comprise” and “comprises”), “having” (and any form thereof such as “have” and “has”), “including” (and any form thereof such as “includes” and “include”) or “containing” (and any form thereof such as “contains” and “contain”) are inclusive or open-ended and do not exclude additional, unrecited elements or method steps.

The term “effective” as used in the specification and claims, means adequate to provide or accomplish a desired, expected, or intended result.

The terms “about” or “approximately” are defined as being close to as understood by one of ordinary skill in the art, and in one non-limiting embodiment the terms are defined to be within 10%, within 5%, within 1%, and in certain aspects within 0.5%.

In certain embodiments, the mitochondria specific binding ligand is an antibody that recognizes mitochondrial specific binding sites. As used herein the term “antibody” includes both intact immunoglobulin molecules as well as portions, fragments, and derivatives thereof, such as, for example, Fab, Fab′, F(ab′)₂, Fv, Fsc, CDR regions, or any portion of an antibody that is capable of binding an antigen or epitope including chimeric antibodies that are bi-specific or that combine an antigen binding domain originating with an antibody with another type of polypeptide. The term antibody includes monoclonal antibodies (mAb), chimeric antibodies, humanized antibodies, as well as fragments, portions, regions, or derivatives thereof, provided by any known technique including but not limited to, enzymatic cleavage and recombinant techniques. The term “antibody” as used herein also includes single-domain antibodies (sdAb) and fragments thereof that have a single monomeric variable antibody domain (V_{H}) of a heavy-chain antibody. sdAb, which lack variable light (V_{L}) and constant light (C_{L}) chain domains are natively found in camelids (V_{H}H) and cartilaginous fish (V_{NAR}) and are sometimes referred to as “Nanobodies” by the pharmaceutical company Ablynx who originally developed specific antigen binding sdAb in llamas. The modifier “monoclonal” indicates the character of the antibody as being obtained from a substantially homogeneous population of antibodies, and is not to be construed as requiring production of the antibody by any particular method.

In other embodiments the mitochondria specific binding ligand is an aptamer that recognizes methylated DNA bases. Aptamers are structurally distinct RNA or DNA oligonucleotides (ODNs) that can mimic protein-binding molecules and exhibit high (nM) binding affinity based on their unique secondary three-dimensional structure conformations and not by pair-wise nucleic acid binding. Aptamers can be selected via high-throughput in vitro methods to bind target molecules. Aptamers are typically approximately 1/10th the molecular weight of antibodies and yet provide complex tertiary, folded structures with sufficient recognition surface areas to rival antibodies.

QDs are fluorescent semiconductor nanoparticles with unique optical properties. QD represent a particular very small size form of semiconductor material in which the size and shape of the particle results in quantum mechanical effects upon light excitation. Generally, larger QDs such as having a radius of 5-6 nm will emit longer wavelengths in orange or red emission colors and smaller QDs such as having a radius of 2-3 nm emit shorter wavelengths in blue and green colors, although the specific colors and sizes depend on the composition of the QD. Quantum Dots shine around 20 times brighter and are many times more photo-stable than any of the conventional fluorescent dyes (like indocyanine green (ICG)). Importantly, QD residence times are longer due to their chemical nature and nano-size. QDs can absorb and emit much stronger light intensities. In certain embodiments, the QD can be equipped with more than one binding tag, forming bi- or tri-specific nano-devices. The unique properties of QDs enable several medical applications that serve unmet needs.

In embodiments presented herein, the QDs are functionalized to present a hydrophilic outer layer or corona that permits use of the QDs in the aqueous environment, such as, for example, in vivo and in vitro applications in living cells. Such QDs are termed water soluble QDs.

In one embodiment the QDs may be surface equipped with a conjugation capable function (COOH, OH, NH₂, SH, azide, alkyne). In one exemplified embodiment, the water soluble non-toxic QD is or becomes carboxyl functionalized. For example, the COOH-QD may be linked to the amine terminus of a targeting antibody using a carbodiimide linking technology employing water-soluble 1-ethyl-3-(-3-dimethylaminopropyl) carbodiimide hydrochloride (EDC). The carboxyl functionalized QD is mixed with EDC to form an active O-acylisourea intermediate that is then displaced by nucleophilic attack from primary amino groups on the monoclonal antibody in the reaction mixture. If desired, a sulfo derivative of N-hydroxysuccinimide (sulfo-NHS) is added during the reaction with the primary amine bearing antibody. With the sulfo-NHS addition, the EDC couples NHS to carboxyls, forming an NHS ester that is more stable than the O-acylisourea intermediate while allowing for efficient conjugation to primary amines at physiologic pH. In either event, the result is a covalent bond between the QD and the antibody. Other chemistries like Suzuki-Miyaura cross-coupling, (succinimidyl 4-(N-maleimidomethyl)cyclohexane-1-carboxylate) (SMCC), or aldehyde based reactions may alternatively be used.

Methods of synthesizing core and core-shell nanoparticles are disclosed, for example, in co-owned U.S. Pat. Nos. 7,867,556, 7,867,557, 7,803,423, 7,588,828, and 6,379,635. The contents of each of the forgoing patents are hereby incorporated by reference, in their entirety. U.S. Pat. Nos. 9,115,097, 8,062,703, 7,985,446, 7,803,423, and 7,588,828, and U.S. Publication Nos. 2010/0283005, 2014/0264196, 2014/0277297 and 2014/0370690, the entire contents of each of which are hereby incorporated by reference, describe methods of producing large volumes of high quality monodisperse quantum dots.

In one embodiment, a core/shell particle is utilized having a central region or “core” of at least one semiconductor composition buried in or coated by one or more outer layers or “shell” of distinctly different semiconductor compositions. As an example, the core may be comprised of an alloy of In, P, Zn and S such as is formed by the description of Example 1 involving molecular seeding of In-based nanoparticles over a ZnS molecular cluster followed by formation of a shell of ZnS.

In still other embodiments, the water soluble QD nanoparticle employed comprises an alloyed semiconductor material having a bandgap value or energy (E_{g}) that increases outwardly by graded alloying in lieu of production of a core/shell QD. The band gap energy (E_{g}), is the minimum energy required to excite an electron from the ground state valence energy band into the vacant conduction energy band.

The graded alloy QD composition is considered “graded” in elemental composition from at or near the center of the particle to the outermost surface of the QD rather than formed as a discrete core overlaid by a discrete shell layer. An example would be an In₁₋ₓP_{1-y}ZnₓS_{y}, graded alloy QD wherein the x and y increase gradually from 0 to 1 from the center of the QD to the surface. In such example, the band gap of the QD would gradually change from that of pure InP towards the center to that of a larger band gap value of pure ZnS at the surface. Although the band gap is dependent on particle size, the band gap of ZnS is wider than that of InP such that the band gap of the graded alloy would gradually increase from an inner aspect of the QD to the surface.

A one-pot synthesis process may be employed as a modification of the molecular seeding process described in Example 1 herein. This may be achieved by gradually decreasing the amounts of indium myristate and (TMS)₃P added to the reaction solution to maintain particle growth, while adding increasing amounts of zinc and sulfur precursors during a process such as is described for generation of the “core” particle of Example 1. Thus, in one example a dibutyl ester and a saturated fatty acid are placed into a reaction flask and degassed with heating. Nitrogen is introduced and the temperature is increased. A molecular cluster, such as for example a ZnS molecular cluster [Et₃NH]₄ [Zn₁₀S₄(SPh)₁₆], is added with stirring. The temperature is increased as graded alloy precursor solutions are added according to a ramping protocol that involves addition of gradually decreasing concentrations of a first semiconductor material and gradually increasing concentrations of a second semiconductor material. For example, the ramping protocol may begin with additions of indium myri state (In(MA)₃) and trimethylsilyl phosphine (TMS)₃P dissolved in a dicarboxylic acid ester (such as for example di-n-butylsebacate ester) wherein the amounts of added In(MA)₃ and (TMS)₃P gradually decrease over time to be replaced with gradually increasing concentration of sulfur and zinc compounds such as (TMS)₂S and zinc acetate. As the added amounts of In(MA)₃ and (TMS)₃P decrease, gradually increasing amounts of (TMS)₂S dissolved in a saturated fatty acid (such as for example myristic or oleic acid) and a dicarboxylic acid ester (such as di-n-butyl sebacate ester) are added together with the zinc acetate. The following reactions will result in the increasing generation of ZnS compounds. As the additions continue, QD particles of a desired size with an emission maximum gradually increasing in wavelength are formed wherein the concentrations of InP and ZnS are graded with the highest concentrations of InP towards a center of the QD particle and the highest concentrations of ZnS on an outer of the QD particle. Further additions to the reaction are stopped when the desired emission maximum is obtained and the resultant graded alloy particles are left to anneal followed by isolation of the particles by precipitation and washing.

A nanoparticle's compatibility with a medium as well as the nanoparticle's susceptibility to agglomeration, photo-oxidation and/or quenching, is mediated largely by the surface composition of the nanoparticle. The coordination about the final inorganic surface atoms in any core, core-shell or core-multi shell nanoparticle may be incomplete, with highly reactive “dangling bonds” on the surface, which can lead to particle agglomeration. This problem is overcome by passivating (capping) the “bare” surface atoms with protecting organic groups, referred to herein as capping ligands or a capping agent. The capping or passivating of particles prevents particle agglomeration from occurring but also protects the particle from its surrounding chemical environment and provides electronic stabilization (passivation) to the particles, in the case of core material. Capping ligands may be but are not limited to a Lewis base bound to surface metal atoms of the outer most inorganic layer of the particle. The nature of the capping ligand largely determines the compatibility of the nanoparticle with a particular medium. Capping ligand may be selected depending on desired characteristics. Types of capping ligands that may be employed include thiol groups, carboxyl, amine, phosphine, phosphine oxide, phosphonic acid, phosphinic acid, imidazole, OH, thio ether, and calixarene groups. With the exception of calixarenes, all of these capping ligands have head groups that can form anchoring centers for the capping ligands on the surface of the particle. The body of the capping ligand can be a linear chain, cyclic, or aromatic. The capping ligand itself can be large, small, oligomeric or polydentate. The nature of the body of the ligand and the protruding side that is not bound onto the particle, together determine if the ligand is hydrophilic, hydrophobic, amphiphilic, negative, positive or zwitterionic.

In many quantum dot materials, the capping ligands are hydrophobic (for example, alkyl thiols, fatty acids, alkyl phosphines, alkyl phosphine oxides, and the like). Thus, the nanoparticles are typically dispersed in hydrophobic solvents, such as toluene, following synthesis and isolation of the nanoparticles. Such capped nanoparticles are typically not dispersible in more polar media. If surface modification of the QD is desired, the most widely used procedure is known as ligand exchange. Lipophilic ligand molecules that coordinate to the surface of the nanoparticle during core synthesis and/or shelling procedures may subsequently be exchanged with a polar/charged ligand compound. An alternative surface modification strategy intercalates polar/charged molecules or polymer molecules with the ligand molecules that are already coordinated to the surface of the nanoparticle. However, while certain ligand exchange and intercalation procedures render the nanoparticle more compatible with aqueous media, they may result in materials of lower quantum yield (QY) and/or substantially larger size than the corresponding unmodified nanoparticle.

For in vivo and in vitro purposes, QDs with low toxicity profiles are desirable if not required. Thus, for some purposes, the quantum dot nanoparticle is preferably substantially free of toxic heavy metals such as cadmium, lead and arsenic (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of heavy metals such as cadmium, lead and arsenic) or is free of heavy metals such as cadmium, lead and arsenic. In one embodiment, reduced toxicity QD that lack heavy metals such as cadmium, lead and arsenic are provided.

The unique properties of QDs enable several potential medical applications including unmet in vitro and in vivo diagnostics in living cells. One of the major concerns regarding the medical applications of QDs has been that the majority of research has focused on QDs containing toxic heavy metals such as cadmium, lead or arsenic. The biologically compatible and water-soluble heavy metal-free QDs described herein can safely be used in medical applications both in vitro and in vivo. In certain embodiments, in vivo compatible water dispersible cadmium-free QDs are provided that have a hydrodynamic size of 10-20 nm (within the range of the dimensional size of a full IgG2 antibody). In one embodiment, the in vivo compatible water dispersible cadmium-free QDs are produced in accordance with the procedures set out in Examples 1 and 2 herein. In certain embodiments, the in vivo compatible water dispersible cadmium-free QDs are carboxyl functionalized and further derivatized with a ligand binding moiety.

Examples of cadmium, lead and arsenic free nanoparticles include nanoparticles comprising semiconductor materials, e.g., ZnS, ZnSe, ZnTe, InP, InSb, AlP, AlS, AlSb, GaN, GaP, GaSb, PbS, PbSe, AgInS₂, CuInS₂, Si, Ge, and alloys and doped derivatives thereof, particularly, nanoparticles comprising cores of one of these materials and one or more shells of another of these materials.

In certain embodiments, non-toxic QD nanoparticles are surface modified to enable them to be water soluble and to have surface moieties that allow derivatization by exposing them to a ligand interactive agent to effect the association of the ligand interactive agent and the surface of the QD. The ligand interactive agent can comprise a chain portion and a functional group having a specific affinity for, or reactivity with, a linking/crosslinking agent, as described below. The chain portion may be, for example, an alkane chain. Examples of functional groups include nucleophiles such as thio groups, hydroxyl groups, carboxamide groups, ester groups, and a carboxyl groups. The ligand interactive agent may, or may not, also comprise a moiety having an affinity for the surface of a QD. Examples of such moieties include thiols, amines, carboxylic groups, and phosphines. If ligand interactive group does not comprise such a moiety, the ligand interactive group can associate with the surface of nanoparticle by intercalating with capping ligands. Examples of ligand interactive agents include C₈₋₂₀ fatty acids and esters thereof, such as for example isopropyl myristate.

It should be noted that the ligand interactive agent may be associated with QD nanoparticle simply as a result of the processes used for the synthesis of the nanoparticle, obviating the need to expose nanoparticle to additional amounts of ligand interactive agents. In such case, there may be no need to associate further ligand interactive agents with the nanoparticle. Alternatively, or in addition, QD nanoparticle may be exposed to ligand interactive agent after the nanoparticle is synthesized and isolated. For example, the nanoparticle may be incubated in a solution containing the ligand interactive agent for a period of time. Such incubation, or a portion of the incubation period, may be at an elevated temperature to facilitate association of the ligand interactive agent with the surface of the nanoparticle. Following association of the ligand interactive agent with the surface of nanoparticle, the QD nanoparticle is exposed to a linking/crosslinking agent and a surface modifying ligand. The linking/crosslinking agent includes functional groups having specific affinity for groups of the ligand interactive agent and with the surface modifying ligand. The ligand interactive agent-nanoparticle association complex can be exposed to linking/crosslinking agent and surface modifying ligand sequentially. For example, the nanoparticle might be exposed to the linking/crosslinking agent for a period of time to effect crosslinking, and then subsequently exposed to the surface modifying ligand to incorporate it into the ligand shell of the nanoparticle. Alternatively, the nanoparticle may be exposed to a mixture of the linking/crosslinking agent and the surface modifying ligand thus effecting crosslinking and incorporating surface modifying ligand in a single step.

In one embodiment quantum dot precursors are provided in the presence of a molecular cluster compound under conditions whereby the integrity of the molecular cluster is maintained and acts as a well-defined prefabricated seed or template to provide nucleation centers that react with the chemical precursors to produce high quality nanoparticles on a sufficiently large scale for industrial application.

The disclosed methods are not limited, however, to molecular cluster methods. Additional methods for preparing the quantum dots include, for example, dual injection methods, aqueous based methods, hot injection methods and seeding methods.

Suitable types of quantum dot nanoparticles useful in the present invention include, but are not limited to, core materials comprising the following types (including any combination or alloys thereof):

IIA-VIB (2-16) material, incorporating a first element from group 2 of the periodic table and a second element from group 16 of the periodic table and also including ternary and quaternary materials and doped materials. Suitable nanoparticle materials include, but are not limited to: MgS, MgSe, MgTe, CaS, CaSe, CaTe, SrS, SrSe, SrTe, BaS, BaSe, and BaTe.

II-V material incorporating a first element from group 12 of the periodic table and a second element from group 15 of the periodic table and also including ternary and quaternary materials and doped materials. Suitable nanoparticle materials include, but are not limited to: Zn₃P₂, Zn₃As₂, Cd₃P₂, Cd₃As₂, Cd₃N₂, and Zn₃N₂.

II-VI material incorporating a first element from group 12 of the periodic table and a second element from group 16 of the periodic table and also including ternary and quaternary materials and doped materials. Suitable nanoparticle materials include, but are not limited to: CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, CdZnSeS, CdZnSeTe, CdHgSeS, CdHgSeTe, CdHgSTe, HgZnSeS, and HgZnSeTe.

III-V material incorporating a first element from group 13 of the periodic table and a second element from group 15 of the periodic table and also including ternary and quaternary materials and doped materials. Suitable nanoparticle materials include, but are not limited to: BP, AlP, AlAs, AlSb; GaN, GaP, GaAs, GaSb; InN, InP, InAs, InSb, AIN, and BN.

III-IV material incorporating a first element from group 13 of the periodic table and a second element from group 14 of the periodic table and also including ternary and quaternary materials and doped materials. Suitable nanoparticle materials include, but are not limited to: B₄C, Al₄C₃, Ga₄C, Si, and SiC.

III-VI material incorporating a first element from group 13 of the periodic table and a second element from group 16 of the periodic table and also including ternary and quaternary materials. Suitable nanoparticle materials include, but are not limited to: Al₂S₃, Al₂Se₃, Al₂Te₃, Ga₂S₃, Ga₂Se₃, GeTe; In₂S₃, In₂Se₃, Ga₂Te₃, In₂Te₃, and InTe.

IV-VI material incorporating a first element from group 14 of the periodic table and a second element from group 16 of the periodic table, and also including ternary and quaternary materials and doped materials. Suitable nanoparticle materials include, but are not limited to: PbS, PbSe, PbTe, Sb₂Te₃, SnS, SnSe, and SnTe.

Nanoparticle material incorporating a first element from any group in the transition metals of the periodic table, and a second element from Group 16 of the periodic table and also including ternary and quaternary materials and doped materials. Suitable nanoparticle materials include, but are not limited to: NiS, CrS, AgS, and I-III-VI materials, for example, CuInS₂, CuInSe₂, CuGaS₂, and AgInS₂.

In a preferred embodiment, the nanoparticle material comprises a II-IV material, a III-V material, a I-III-VI material, and any alloy or doped derivative thereof.

The term doped nanoparticle for the purposes of specifications and claims refers to nanoparticles of the above and a dopant comprising one or more main group or rare earth elements, this most often is a transition metal or rare earth element, such as but not limited to zinc sulfide with manganese, such as ZnS nanoparticles doped with Mn⁺.

In one embodiment, the quantum dot nanoparticle is substantially free of heavy metals such as cadmium (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of heavy metals such as cadmium) or is free of heavy metals such as cadmium.

For in vivo applications, heavy metal-free semi-conductor nanoparticles such as In-based quantum dots, for example, InP quantum dots and their alloys and doped derivatives are preferred.

In an embodiment, any of the quantum dot nanoparticles described herein include a first layer including a first semiconductor material provided on the nanoparticle core. A second layer including a second semiconductor material may be provided on the first layer.

### Synthesis

The following synthesis steps may be used for conjugation. Linkers may be used to form an amide group between the carboxyl functions on the nanoparticles and the amine end groups on the methylation specific binding ligand. Known linkers, such as a thiol anchoring groups directly on the inorganic surface of the quantum dot nanoparticle can be used. Standard coupling conditions can be employed and will be known to a person of ordinary skill in the art. For example, suitable coupling agents include, but are not limited to, carbodiimides, such as dicyclohexylcarbodiimide (DCC), diisopropylcarbodiimide (DIC), and 1-(3-dimethylaminopropyl)-3-ethylcarbodiimide hydrochloride (EDC). In one embodiment, the coupling agent is EDC.

In an example, the quantum dot nanoparticles bearing a carboxyl end group and a functionalized ligand may be mixed in a solvent. A coupling agent, such as EDC, may be added to the mixture. The reaction mixture may be incubated. The crude functionalized ligand nanoparticle conjugate may be subject to purification and/or isolated.

Standard solid state purification method may be used. Several cycles of filtering and washing with a suitable solvent may be necessary to remove excess unreacted functionalized ligand and/or EDC.

The following examples are include for the sake of completeness of disclosure and to illustrate the methods of making the compositions and composites of the present invention as well as to present certain characteristics of the compositions. In no way are these examples intended to limit the scope or teaching of this disclosure.

### EXAMPLE 1

### Synthesis of Non-Toxic Quantum Dots

A molecular seeding process was used to generate non-toxic Quantum Dots (QD). Briefly, the preparation of non-functionalized indium-based quantum dots with emission in the range of 500-700 nm was carried out as follows: Dibutyl ester (approximately 100 ml) and myristic acid (MA) (10.06 g) were placed in a three-neck flask and degassed at ˜70° C. under vacuum for 1 h. After this period, nitrogen was introduced and the temperature was increased to ˜90° C. Approximately 4.7 g of a ZnS molecular cluster [Et₃NH]₄ [Zn₁₀S₄(SPh)₁₆] was added, and the mixture was stirred for approximately 45 min. The temperature was then increased to ˜100° C., followed by the drop-wise additions of In(MA)₃ (1M, 15 ml) followed by trimethylsilyl phosphine (TMS)₃P (1M, 15 ml). The reaction mixture was stirred while the temperature was increased to ˜140° C. At 140° C., further drop-wise additions of indium myristate (In(MA)₃) dissolved in di-n-butylsebacate ester (1M, 35 ml) (left to stir for 5 min) and (TMS)₃P dissolved in di-n-butylsebacate ester (1M, 35 ml) were made. The temperature was then slowly increased to 180° C., and further dropwise additions of In(MA)₃ (1M, 55 ml) followed by (TMS)₃P (1M, 40 ml) were made. By addition of the precursor in this manner, indium-based particles with an emission maximum gradually increasing from 500 nm to 720 nm were formed. The reaction was stopped when the desired emission maximum was obtained and left to stir at the reaction temperature for half an hour. After this period, the mixture was left to anneal for up to approximately 4 days (at a temperature ˜20-40 ° C. below that of the reaction). A UV lamp was also used at this stage to aid in annealing.

The particles were isolated by the addition of dried degassed methanol (approximately 200 ml) via cannula techniques. The precipitate was allowed to settle and then methanol was removed via cannula with the aid of a filter stick. Dried degassed chloroform (approximately 10 ml) was added to wash the solid. The solid was left to dry under vacuum for 1 day. This procedure resulted in the formation of indium-based nanoparticles on ZnS molecular clusters. In further treatments, the quantum yields of the resulting indium-based nanoparticles were further increased by washing in dilute hydrofluoric acid (HF). The quantum efficiencies of the indium-based core material ranged from approximately 25%-50%. This composition is considered an alloy structure comprising In, P, Zn and S.

Growth of a ZnS shell: A 20 ml portion of the HF-etched indium-based core particles was dried in a three-neck flask. 1.3 g of myristic acid and 20 ml di-n-butyl sebacate ester were added and degassed for 30 min. The solution was heated to 200° C., and 2 ml of 1 M (TMS)₂S was added drop-wise (at a rate of 7.93 ml/h). After this addition was complete, the solution was left to stand for 2 min, and then 1.2 g of anhydrous zinc acetate was added. The solution was kept at 200° C. for 1 hr and then cooled to room temperature. The resulting particles were isolated by adding 40 ml of anhydrous degassed methanol and centrifuging. The supernatant liquid was discarded, and 30 ml of anhydrous degassed hexane was added to the remaining solid. The solution was allowed to settle for 5 h and then centrifuged again. The supernatant liquid was collected and the remaining solid was discarded. The quantum efficiencies of the final non-functionalized indium-based nanoparticle material ranged from approximately 60%-90% in organic solvents.

### EXAMPLE 2

### Water Soluble Surface Modified QDs

Provided herein is one embodiment of a method for generating and using melamine hexamethoxymethylmelamine (HMMM) modified fluorescent nanoparticles as drug delivery vehicles. The unique melamine-based coating presents excellent biocompatibility, low toxicity and very low non-specific binding. These unique features allow a wide range of biomedical applications both in vitro and in vivo.

One example of preparation of a suitable water soluble nanoparticle is provided as follows: 200 mg of cadmium-free quantum dot nanoparticles with red emission at 608 nm having as a core material an alloy comprising indium and phosphorus with Zn-containing shells as described in Example 1 was dispersed in toluene (1 ml) with isopropyl myristate (100 microliters). The isopropyl myristate is included as the ligand interactive agent. The mixture was heated at 50° C. for about 1-2 minutes then slowly shaken for 15 hours at room temperature. A toluene solution (4 ml) of hexamethoxymethylmelamine (HMMM) (CYMEL 303, available from Cytec Industries, Inc., West Paterson, N.J.) (400 mg), monomethoxy polyethylene oxide (CH₃O-PEG₂₀₀₀-OH) (400 mg), and salicylic acid (50 mg) was added to the nanoparticle dispersion. The salicylic acid that is included in the functionalization reaction plays three roles, as a catalyst, a crosslinker, and a source for COOH. Due in part to the preference of HMMM for OH groups, many COOH groups provided by the salicylic acid remain available on the QD after crosslinking.

HMMM is a melamine-based linking/crosslinking agent having the following structure:

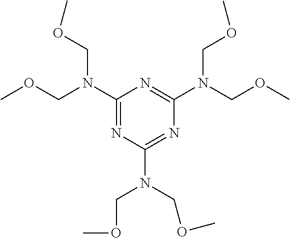

HMMM can react in an acid-catalyzed reaction to crosslink various functional groups, such as amides, carboxyl groups, hydroxyl groups, and thiols.

The mixture was degassed and refluxed at 130° C. for the first hour followed by 140° C. for 3 hours while stirring at 300 rpm with a magnetic stirrer. During the first hour a stream of nitrogen was passed through the flask to ensure the removal of volatile byproducts generated by the reaction of HMMM with nucleophiles. The mixture was allowed to cool to room temperature and stored under inert gas. The surface-modified nanoparticles showed little or no loss in fluorescence quantum yield and no change in the emission peak or full width at half max (FWHM) value, compared to unmodified nanoparticles. An aliquot of the surface-modified nanoparticles was dried under vacuum and deionized water was added to the residue. The surface-modified nanoparticles dispersed well in the aqueous media and remained dispersed permanently. In contrast, unmodified nanoparticles could not be suspended in the aqueous medium. The fluorescence quantum yield of the surface-modified nanoparticles according to the above procedure is 40-50%. In typical batches, a quantum yield of 47%±5% is obtained.

In another embodiment, cadmium-free quantum dot nanoparticles (200 mg) with red emission at 608 nm were dispersed in toluene (1 ml) with cholesterol (71.5 mg). The mixture was heated at 50° C. for about 1-2 minutes then slowly shaken for 15 hours at room temperature. A toluene solution (4 ml) of HMMM (Cymel 303) (400 mg), monomethoxy polyethylene oxide (CH₃O-PEG₂₀₀₀-OH) (400 mg), guaifenesin (100 mg), dichloromethane (DCM) (2 mL) and salicylic acid (50 mg) was added to the nanoparticle dispersion.

As used herein the compound “guaifenesin” has the following chemical structure:

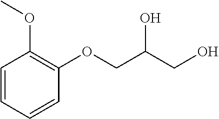

As used herein the compound “salicylic acid” has the following chemical structure:

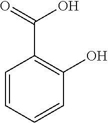

The mixture was degassed and refluxed at 140° C. for 4 hours while stirring at 300 rpm with a magnetic stirrer. As with the prior procedure, during the first hour a stream of nitrogen was passed through the flask to ensure the removal of volatile byproducts generated by the reaction of HMMM with nucleophiles. The mixture was allowed to cool to room temperature and stored under inert gas. An aliquot of the surface-modified nanoparticles was dried under vacuum and deionized water was added to the residue. The pH of the solution was adjusted to 6.5 using a 100 mM KOH solution and the excess non reacted material was removed by three cycles of ultrafiltration using Amicon filters (30 kD). The final aqueous solution was kept refrigerated until use.

It is noteworthy that traditional methods for modifying nanoparticles to increase their water solubility (e.g., ligand exchange with mercapto-functionalized water soluble ligands) are ineffective under mild conditions to render the nanoparticles water soluble. Under harsher conditions, such as heat and sonication, the fraction that becomes water soluble has very low quantum yield (QY <20%). The instant method, in contrast, provides water soluble nanoparticles with high quantum yield. As defined herein, a high quantum yield is equal to or greater than 40%. In certain embodiments, a high quantum yield is obtained of equal to or greater than 45%. The surface-modified nanoparticles prepared as in this example also disperse well and remain permanently dispersed in other polar solvents, including ethanol, propanol, acetone, methylethylketone, butanol, tripropylmethylmethacrylate, or methylmethacrylate.

### EXAMPLE 3

### Water Soluble QD Including Targeting Ligands

In certain embodiments, the water soluble QD is modified to include targeting ligands that are added to the QD. Thus, in one embodiment quantum dot nanoparticles are synthesized that are non-toxic and water soluble (biocompatible) and are surface equipped with a conjugation capable function (COOH, OH, NH₂, SH, azide, alkyne). By virtue of the functional groups that can be added to the QD, such as for example the COOH functional group provided in Example 2 herein, the QD can be modified to include a targeting ligand that allows the QD to selectively identify mitochondria in samples, cells and tissues. The targeting ligand modified QD is the irradiated and emits light for detection.

In one exemplified embodiment, the water soluble non-toxic QD is or becomes carboxyl functionalized. The COOH-QD is linked to the amine terminus of a nitochondrial targeting moiety such as a specific antibody using a chemical method such as for example a carbodiimide linking technology employing water-soluble 1-ethyl-3-(-3-dimethylaminopropyl) carbodiimide hydrochloride (EDC). The carboxyl functionalized QD is mixed with EDC to form an active O-acylisourea intermediate that is then displaced by nucleophilic attack from primary amino groups on the monoclonal antibody in the reaction mixture. If desired, a sulfo derivative of N-hydroxysuccinimide (sulfo-NHS) is added during the reaction with the primary amine bearing antibody. With the sulfo-NHS addition, the EDC couples NHS to carboxyls, forming an NHS ester that is more stable than the O-acylisourea intermediate while allowing for efficient conjugation to primary amines at physiologic pH. In either event, the result is a covalent bond between the QD and the antibody. Other chemistries like Suzuki-Miyaura cross-coupling, succinimidyl 4-(N-maleimidomethyl) cyclohexane-1-carboxylate (SMCC), or aldehyde based reactions may alternatively be used.

In one embodiment, non-toxic water soluble quantum dots are chemically attached to an antibody directed to mitochondrial binding. Suitable methylation specific binding ligands include, but are not limited to, anti mitochondria antibody sold by tebu-bio (cat. No. 909-301-D79), Anti HSP60 (T547) sold by tebu-bio (cat nos. BS1179-50 ul (50 ul) and BS1179-100 ul (100 ul)), Anti SOD1 sold by tebu-bio (cat no. MAB10394), Anti Grp75 clone S19-2 sold by tebu-bio (cat no. MAB6629), Anti Cytochrome c (H19) sold by tebu-bio (cat nos. BS1089-50 ul (50 ul) and BS1089-100 ul (100 ul)), triphenylphosphonium (TPP), and any combination thereof.

Covalent conjugation of in vivo compatible water dispersible cadmium free QD with mitochondrial specific binding ligands: In Eppendorf tubes, 1 mg carboxyl-functionalised, water-soluble quantum dots are mixed with 100 μl MES activation buffer (i.e. 25 μl of 40 mg/ml stock into 100 μl MES). The MES buffer is prepared as a 25 mM solution (2-(N-morpholino) ethanesulfonic acid hemisodium salt (MES), Sigma Aldrich) in deionized (DI) water, pH 4.5. To this, 33 μl of a fresh EDC solution (30 mg/ml stock in DI water, 1-ethyl-3-(3-dimethylaminopropyl) carbodiimide hydrochloride (EDC), Fisher Scientific) is added and the solution is mixed. 4 μl of fresh sulfo-NHS (100 mg/ml stock, ThermoFisher Scientific, in DI water) is added and mixed. NanoSep 300K filters (PALL NanoSep 300K Omega ultrafilters) are pre-wetted in 100 μl MES. The MES/EDC/Sulfo-NHS/QD solution is added to the NanoSep 300K filter and is topped off with sufficient MES. The filter is centrifuged at 5000 rpm/15 min. The dots are re-dispersed in 50 μl activation buffer and are transferred to an Eppendorf tube containing 10 μl of mitochondrial specific ligand. The solution is mixed well and incubated at room temperature overnight (around 16-18 hours). The solution is quenched with 16 μl of 6-amino caproic acid (6AC) (19.7 mg/100 mM). Note that quenching could be alternatively conducted with other compounds having a primary amine, but 6AC is selected for this embodiment because it has a COOH and can maintain the colloidal stability of the product. The solution is transferred to a pre-whetted Nanosep 300K filter (100 μl 1× PBS) and is topped-up to the 500 μl line with 1× PBS. Excess SAV is removed by three cycles of ultrafiltration using Nanosep 300K filters and 1× PBS buffer. Each cycle of centrifugation is run at 5000 rpm for 20 min with re-dispersal with ˜400 ul of 1× PBS after each cycle. The final concentrate is re-dispersed in 100 μl PBS.

### EXAMPLE 4

### In Vitro Application

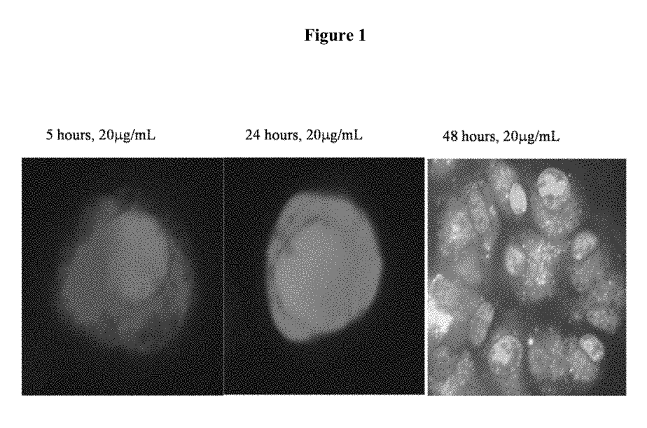

Since the COX enzyme has light absorption peaks at 620, 680, 760 and 820 nm, the quantum dot emission can be tuned to match one of the COX absorptive peaks. In this example, 620 nm emissive quantum dot nanoparticles were used. Cultivated cancer cells were incubated with water soluble 620 nm emissive quantum dot nanoparticles at a range of concentrations (1-20 μg/mL). After a predetermined time, fluorescent microscopy images were taken to detect the internalization of the dots. As can be seen in FIG. 1, the quantum dot nanoparticles were internalized into the cytoplasmic structure. The QDs are then irradiated, and the subsequent emission from the internalized QDs are used to stimulate COX and modulate COX activity or release from the mitochondria in the cultivated cancer cell culture.

Studies have shown that the enzyme cytochrome c oxidase (COX) is considered as a photo-acceptor and photo-signal transducer in the region of visible and near infra-red (NIR). Photo-irradiation by internalized QDs causes an increase in COX activity that leads to a cascade of reactions which can alter cellular homeostasis and increase the production of adenosine triphosphate (ATP), reactive oxygen species (ROS), nitric oxide (NO), and intracellular calcium (iCa2+). In vitro applications of photo-emitting internalized QDs provides better understanding of the role COX plays in cell function and disease.

### EXAMPLE 5

### In Vivo Application—Homeostasis

Depending on the therapeutic objective and the method of administration, the quantum dot nanoparticles can be used as simple naked dots injected directly to an area of concern in a mammal (for example, a targeted area or organ). Photo-irradiation by internalized QDs sufficient to increase COX activity is believed to be responsible for low-intensity light therapy by regulating homeostasis (e.g., increasing the production of adenosine triphosphate (ATP), reactive oxygen species (ROS), nitric oxide (NO), and/or intracellular calcium (iCa2+)). See Tafur et al., Low-Intensity Light Therapy: Exploring the Role of Redox Mechanisms, Photomed Laser Surg. 2008 August; 26(4): 323-328. The photo-irradiation by internalized QDs is used to target the area of concern in a mammal without subjecting other areas to photo-irradiation. The internal photo-irradiation by the QDs is used to modulate the process of inflammation repair, wound healing, and soft tissue repair.

The QDs can also be administered by subcutaneous, intramuscular, intradermal, or intravenous routes. Equipping QDs with tissue specific tags (in accordance with Example 3 discussed above) to ensure specific delivery in a mammal the QDs target the area of concern (for example, a particular organ in a mammal).

### EXAMPLE 5

### In Vivo Application—Apoptosis

Depending on the therapeutic objective and the method of administration, the quantum dot nanoparticles can be used as simple naked dots injected directly to an area of concern in a mammal (for example, a targeted area or organ). Photo-irradiation by internalized QDs causes an increase in COX activity; an overexpression of COX is believed to trigger pathways to apoptosis by reaching the cytoplasm of the cell within which the mitochondria is present. See, Boehning D, et al., Cytochrome c binds to inositol (1,4,5) trisphosphate receptors, amplifying calcium-dependent apoptosis. Nature Cell Biology. 5 (12): 1051-61 (December 2003). The photo-irradiation by internalized QDs is used to target the area of concern in a mammal without subjecting other areas to photo-irradiation. The internal photo-irradiation by the QDs is used to initiate cell death (apoptosis) in undesired cells, such as, for example tumor cells. The photo-irradiation results in depolarization of mitochondrial membrane, resulting in the release of COX into the cytoplasm of the cell within which the mitochondria is present.

The QDs can also be administered by subcutaneous, intramuscular, intradermal, or intravenous routes. Equipping QDs with tissue specific tags (in accordance with Example 3 discussed above) to ensure specific delivery in a mammal the QDs target the area of concern (for example, a particular organ in a mammal).

All publications, patents and patent applications cited herein are hereby incorporated by reference as if set forth in their entirety herein. These and other advantages of the present invention will be apparent to those skilled in the art from the foregoing specification. Accordingly, it is to be recognized by those skilled in the art that changes or modifications may be made to the above-described embodiments without departing from the broad inventive concepts of the invention. It is to be understood that this invention is not limited to the particular embodiments described herein, but is intended to include all changes and modifications that are within the scope and spirit of the invention.

What is claimed is:

1. A nanoparticle conjugate comprising:
a quantum dot comprising:
a core semiconductor material, and
an outer layer,
wherein the outer layer comprises a functionalization organic coating linked to a functionalized ligand that allows metabolic activities in mitochondria to be modulated.

2. The nanoparticle conjugate of claim 1, wherein the nanoparticle conjugate is surface modified to enhance accumulation of the nanoparticle conjugate in mitochondria.

3. The nanoparticle conjugate of claim 1, wherein the nanoparticle conjugate is coated with a mitochondriotopic ligand.

4. The nanoparticle conjugate of claim 1, wherein mitochondriotopic ligand is triphenylphosphonium, or a derivative thereof.

5. The nanoparticle conjugate of claim 4, wherein the nanoparticle comprises a II-VI material, a III-V material, a I-III-VI material or any alloy thereof.

6. The nanoparticle conjugate of claim 5, wherein the functionalized ligand is a hexokinase (HK) inhibitor.

7. The nanoparticle conjugate of claim 6, wherein the hexokinase (HK) inhibitor is 2-deoxyglucose (2DG).

8. The nanoparticle conjugate of claim 7, further comprising a cellular uptake enhancer, a tissue penetration enhancer, or a combination thereof.

9. A process for preparing a ligand nanoparticle conjugate comprising coupling a nanoparticle with a functionalized ligand that allows metabolic activities in mitochondria to be modulated, wherein the nanoparticle comprises a quantum dot, a core semiconductor material, and an outer layer, wherein the outer layer comprises a carboxyl group.

10. The process of claim 9, further comprising purifying the ligand nanoparticle conjugate.

11. The process of claim 10, further comprising isolating the ligand nanoparticle conjugate.

12. The process of claim 11, wherein the coupling step is conducted in the presence of a couple agent.

13. The process of claim 12, wherein the coupling agent is 1-(3-dimethylaminopropyl)-3-ethylcarbodiimide hydrochloride.

14. A method of manipulating mitochondrial function comprising
i) contacting a ligand nanoparticle conjugate according to claim 1 with mitochondria; and
ii) exciting the quantum dot nanoparticle with a light source.

15. The method of claim 14, whereby a voltaic current stimulates the release of cytochrome c oxidase.

16. The method of claim 15, whereby the photons generated photo-modulate the activity of cytochrome c oxidase.

17. A method of modulating the activity of cytochrome c oxidase comprising
i) contacting a ligand nanoparticle conjugate according to claim 1 with mitochondria;
ii) exciting the quantum dot nanoparticle with a light source; and
iii) generating photo-irradiation from the quantum dot nanoparticle.

18. The method of claim 17, whereby the voltaic current stimulates the release of cytochrome C oxidase.

19. The method of claim 18, whereby the release of cytochrome c oxidase initiates cell death.

20. A method of depolarizing a mitochondrial membrane comprising
i) contacting a ligand nanoparticle conjugate according to claim 1 with mitochondria; and
ii) exciting the quantum dot nanoparticle with a light source; and
iii) generating photo-irradiation from the quantum dot nanoparticle.

21. The method of claim 20, wherein the method induces apoptosis.

22. The method of claim 20, wherein mitochondrial membrane is depolarized via a photovoltaic effect.

23. A method of inhibiting glycolysis comprising
i) contacting a ligand nanoparticle conjugate according to claim 1 with mitochondria; and
ii) exciting the quantum dot nanoparticle with a light source; and
iii) generating photo-irradiation from the quantum dot nanoparticle.
